SEQUENCE LISTING (2 sequences)

SEQ ID NO: 1 - DNA, 34 bp, synthetic construct
  source 1..34: mol_type = other DNA
ttttttccataacctttccacataccgcagacgg

SEQ ID NO: 2 - DNA, 28 bp, synthetic construct
  source 1..28: mol_type = other DNA
ccgtctgcggtatgtggaaaggttatgg

(19) United States
(12) Patent Application Publication
(10) Pub. No.: US 2023/0384257 A1
(43) Pub. Date: Nov. 30, 2023

(54) GROOVE-TYPE FIELD EFFECT TRANSISTOR BIOSENSOR BASED ON ATOMIC LAYER DEPOSITED SEMICONDUCTOR CHANNEL

(71) Applicant: FUZHOU UNIVERSITY, Fuzhou (CN)

(72) Inventors: Diaoyang LI, Fuzhou (CN); Benhui LIN, Fuzhou (CN); Mingwei WANG, Fuzhou (CN); Bo WANG, Fuzhou (CN)

(73) Assignee: FUZHOU UNIVERSITY, Fuzhou (CN)

(21) Appl. No.: 18/173,101

(22) Filed: Feb. 23, 2023

(30) Foreign Application Priority Data
May 24, 2022 (CN) 202210567840.0

(51) Int. Cl. G01N 27/414; G01N 33/569; G01N 33/543

(52) U.S. Cl. CPC G01N 27/4145; G01N 27/4146; G01N 33/56983; G01N 33/5438

(57) ABSTRACT

A groove-type field effect transistor biosensor based on an atomic layer deposited semiconductor channel is provided. By utilizing the characteristics of excellent step coverage and precise control of an atomic-level film thickness of Atomic Layer Deposition, a high-k dielectric and an indium tin oxide (ITO) semiconductor are sequentially deposited on the three-dimensional groove structure to prepare the biosensor with three-dimensional groove structure field effect transistor. A device with the three-dimensional groove structure can overcome the influence of Debye Screening Effect, achieve a longer Debye length than that with a planar structure, and can detect low-concentration disease markers in high ionic strength solutions, and it has the advantages of high sensitivity and rapid detection, and shows a broad application prospect in the fields of instant detection, invitro diagnosis, biochemical analysis, etc.

### CROSS REFERENCE TO THE RELATED APPLICATIONS

This application is based upon and claims priority to Chinese Patent Application No. 202210567840.0, filed on May 24, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBYC070_Sequence_Listing. xml, created on Feb. 3, 2023, and is 2,741 bytes in size.

### TECHNOLOGY FIELD

The invention belongs to the technical field of biosensors. The invention particularly provides a groove-type field effect transistor biosensor based on an atomic layer deposition oxide semiconductor channel.

### BACKGROUND

A field effect transistor biosensor (FET biosensor) has become one of the most promising biological detection technologies due to its advantages of being label-free, high sensitivity, easy integration, etc. At present, one-dimensional semiconductor materials such as carbon nanotubes, silicon nanowires, and the like, two-dimensional semiconductor materials such as graphene, molybdenum disulfide, and the like are widely used for constructing field effect transistor biosensors, but the problems of great difficulty in material preparation and device manufacturing processes exist, and the practical application of the field effect transistor biosensors is greatly limited. Recent studies have shown that indium tin oxide (ITO) has a high carrier concentration, and when its film thickness is greatly reduced, the carrier transport performance will not be affected. In addition, a material preparation process of ITO is completely compatible with an existing mainstream thin film growth process, and a device preparation process of ITO FET is also completely compatible with a complementary metal oxide semiconductor (CMOS) process, so that ITO will be the most potential semiconductor channel material for mass production of FET biosensors compared with nanowires, nanotubes, nanosheets and other materials.

Although FET biosensor is one of the most promising biological detection technologies, due to the existence of Debye Screening, when detecting biological samples with high ionic strength such as blood, serum, urine and sweat, the sensitivity of the FET biosensor will be greatly deteriorated or the target molecules will not be detected at all. At present, the methods to overcome Debye Screening are as follows. (1) Dilution method. Biological samples with high ionic strength are diluted with a low-ionic-strength buffer solution or deionized water. This method is simple and convenient, but excessive dilution will cause a serious salt dissolution effect of protein, making the effect of specific binding become worse and affecting detection results. (2) A desalinating method. Target molecules are purified from biological samples with high ionic strength to remove irrelevant biomolecules and ions. This method is complicated in process and time-consuming, and cannot meet the requirements of instant detection. (3) A sensing surface is modified with a polyethylene glycol (PEG) permeable polymer layer. PEG is widely used in biosensors for antifouling to increase specificity, and also used to modify the sensing surface of the FET biosensor, thus overcoming Debye shielding to some extent. However, PEG is easily oxidized, and PEG cannot permeate all biomolecules by permeating a polymer layer, so that it is possible to block target molecules. (4) Optimization of a device structure. For example, a flat graphene channel is deformed into wrinkled graphene, thus forming an “electric hot spot” in a recess and extending the Debye length. However, compared with flat graphene, the preparation process of the wrinkled graphene is more difficult. Besides the four main methods listed, there are an antibody cleaving method, a double electrode layer destruction method, and the like.

### SUMMARY

In order to overcome the defects in the prior art, the invention provides an indium tin oxide field effect transistor biosensor with a groove-type channel. This device structure overcomes the influence of Debye Screening, and can detect low-concentration disease markers in a high ionic strength solution.

To achieve the purpose above, the invention adopts the following technical solution.

A groove-type field effect transistor biosensor based on an atomic layer deposited semiconductor channel, wherein the biosensor comprises a substrate, a plurality of grooves are provided at a surface of the substrate in a spaced manner, a high-k dielectric layer is provided on the substrate, an ITO channel layer is provided on the high-k dielectric layer, a source electrode and a drain electrode are provided at two ends of the ITO channel layer, and insulating layers are provided on the source electrode and the drain electrode.

Preferably, the substrate is a silicon wafer, a depth of the groove is 10-200 nm, a convex width is 40-200 nm, and a width of the groove is 40-200 nm.

Preferably, the substrate is subjected to photoresist spin coating, baking, exposure, development, fixing, dry etching, photoresist stripping processes, or subjected to photoresist spin coating, baking, nano-imprinting, dry etching, and photoresist stripping processes, so that the flat silicon wafer is prepared into a silicon wafer substrate with several grooves provided on its surface in a spaced manner.

Preferably, the high-k dielectric layer is HfO₂, Al₂O₃, SiO₂ or SiNₓ, which is prepared by Atomic Layer Deposition, with a thickness of 5-10 nm.

Preferably, the ITO channel layer is prepared by Atomic Layer Deposition, with a thickness of 10-20 nm, and the ITO channel layer is concave-convex, with a groove depth of 10-200 nm, a groove width of 20-300 nm, and a convex ITO width of 10-100 nm.

Preferably, the source electrode and the drain electrode are one of Au, Ni/Au, Ni/Au/Ni, and are formed on two ends of ITO through a series of micro-nano processing processes such as photoresist spin coating, baking, exposure, development, fixing, oxygen plasma stripping, metal evaporation and lift-off or a shutter mask evaporation process. The insulating layers are SU-8, polymethyl methacrylate (PMMA), SiO₂ or SiNₓ.

Preferably, a surface of the concave-convex ITO channel layer is modified with biological probes to specifically capture target biomolecules. Specifically, firstly, a surface of the ITO channel layer is treated with oxygen plasma to form hydroxyl groups on the surface of the ITO channel layer, then the ITO channel layer is modified with amino groups, biological probes such as DAN and antibody are immobilized to the surface of the ITO channel layer, and dropwise added to the surface of the ITO channel layer modified with the amino groups, so that chemically active groups in DNA and antibody react with the amino groups, that is, the biological probes such as DNA and antibody are immobilized to the surface of ITO.

The invention has the advantages that: (1) the ITO growth and device preparation process is completely compatible with the existing silicon-based CMOS process, and the mass production potential is huge. (2) The preparation process of the groove-type ITO is compatible with the silicon-based CMOS process. (3) When detecting samples with high ionic strength such as blood, urine, and sweat, although the biomolecules exceed the Debye length, the influence of the Debye length can be effectively overcome if the biomolecules are within the Debye length of a groove-type ITO sidewall. The groove-type ITO can effectively overcome the influence of the Debye length, and further increase the potential of the field effect transistor in clinical sample detection.

### BRIEF DESCRIPTION OF THE DRAWINGS

FIG. 1 is a schematic structural diagram of a groove-type field effect transistor biosensor based on an atomic layer deposited semiconductor channel;

FIG. 2 is a schematic structural diagram of an ITO channel layer; H is a depth of a groove, W is a width of the groove, and L is the width of a convex ITO;

FIG. 3 is a schematic diagram of the principle of antigen detection by the groove-type field effect transistor biosensor based on the atomic layer deposited semiconductor channel, and 34 represents a complex formed from bioprobe antibody and target antigen, and the antibody specifically captures antigen. When detecting samples with high ionic strength such as blood, urine and sweat, although the length of the antibody or antibody-antigen complex exceeds the Debye length, the influence of the Debye length can be effectively overcome if the antibody or antibody-antigen complex is within the Debye length of the groove-type ITO sidewall;

FIG. 4 is a schematic diagram of the principle of DNA detection by the groove-type field effect transistor biosensor based on the atomic layer deposited semiconductor channel, and 44 represents a double-stranded DNA formed by the specific binding of biological probe DNA and target DNA; when detecting samples with high ionic strength such as blood, urine and sweat, although the length of the double-stranded DNA exceeds the Debye length, the influence of the Debye length can be effectively overcome if the double-stranded DNA is within the Debye length on a groove-type ITO side wall;

FIGS. 5A-5B show signal responses of an indium tin oxide field effect transistor biosensor with groove-type and planar channels to target DNA;

FIG. 6 shows signal responses of an indium tin oxide field effect transistor biosensor with groove-type and planar channels to IgG;

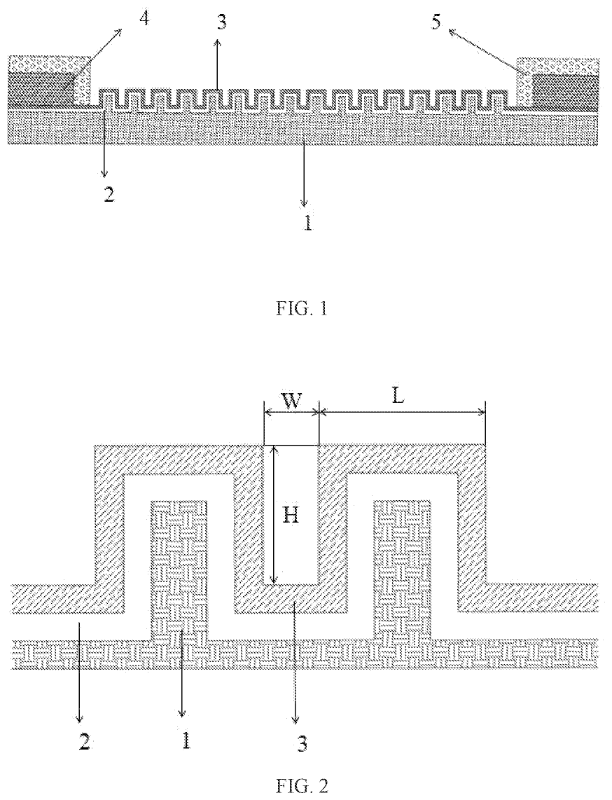

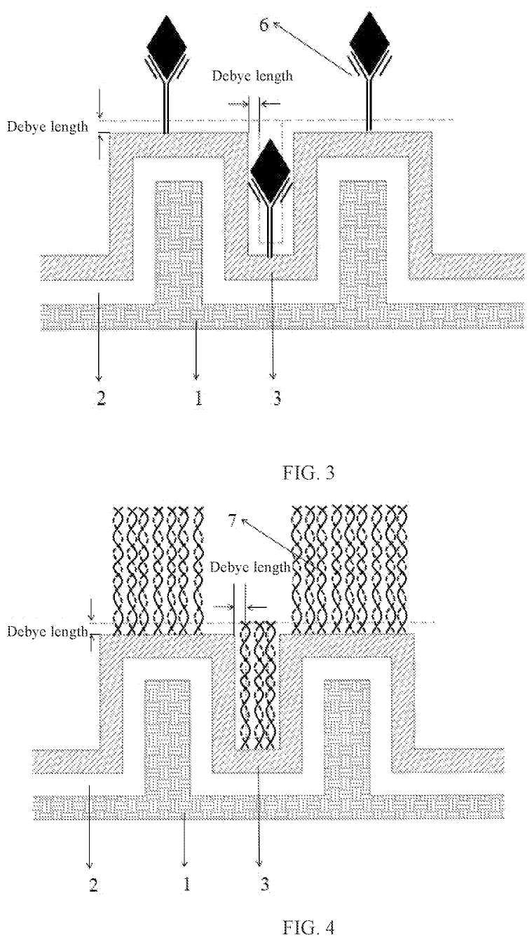

In the figures, 1, silicon substrate; 2, high-κ dielectric layer; 3, ITO; 4, source-drain electrode; 5, insulating layer; 6, complex formed from bioprobe antibody and target antigen; 7, double-stranded DNA formed by specific binding of bioprobe DNA and target DNA.

### DETAILED DESCRIPTION OF THE EMBODIMENTS

In order to make the content of the present invention more understandable, the technical solutions of the present invention are further described below with reference to specific embodiments.

Embodiment 1: DNA detection
DNA probe sequence:
COOH-5′-TTTTTTCCATAACCTTTCCACATACCGCAGACGG-3′,
as shown in SEQ ID NO: 1;
DNA target sequence:
5′-CCGTCTGCGGTATGTGGAAAGGTTATGG-3′,
as shown in SEQ ID NO: 2;

Said DNA probe and DNA target were synthesized by Shanghai Sangon Biotech Co., Ltd.

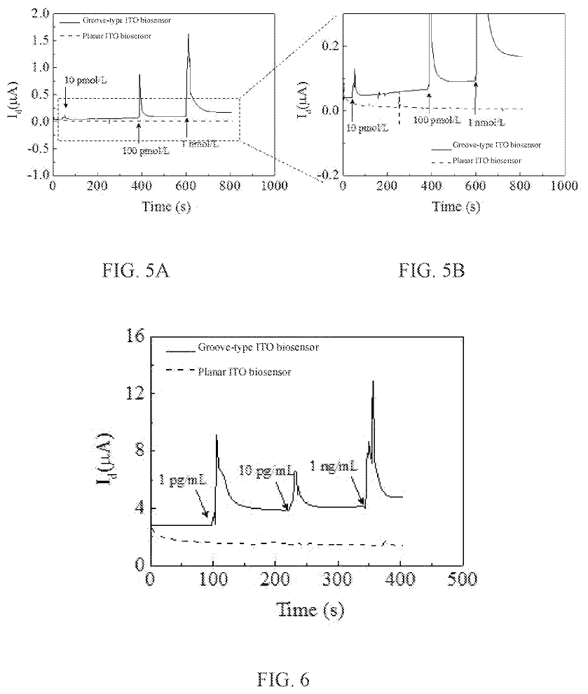

A groove-type field effect transistor biosensor based on an atomic layer deposited semiconductor channel comprises a substrate 1, wherein a plurality of grooves are provided at a surface of the substrate 1 in a spaced manner, a high-k dielectric layer 2 is provided on the substrate 1, an ITO channel layer 3 is provided on the high-k dielectric layer 2, source and drain electrodes 4 are provided at two ends of the ITO channel layer 3, and insulating layers 5 are provided on the source and drain electrodes 4; and the following is a preparation method of the sensor.
1. A substrate silicon is cleaned. The silicon wafer is P-type boron-doped (B), and its resistance is less than 0.005 ohm. A standard RCA1 cleaning process is used to remove particles, organic substances and the like on the substrate. After being cleaned, the substrate is blow-dried with high purity nitrogen for use.
2. A concave-convex silicon surface is defined by the process steps of photoresist leveling, baking, exposure, development, fixing, photoresist stripping, etc. (1) First, on the basis of step 1, spin coating with a ZEP 520A electron beam photoresist is performed with spin-coating parameters of 500 RPM/5 s and 4000 RPM/60 s, and then baking is performed at 180° C. for 3 min. (2) A groove area is defined by an electron beam exposure system. (3) Development: with a developer being xylene, development is performed for 70 s, then fixing with IPA is performed for 30 s, and blow-drying with nitrogen is performed.
3. Silicon is subjected to dry etching. (1) etching process parameters are as follows: the chuck temperature is 10° C., the pressure is 19 mtorr, the radio-frequency power is 300W, the bias voltage is 300V, the flow ratio of sulfur hexafluoride/tetracarbon octafluoride/argon gas is equal to 20/50/30 sccm, and etching is performed for 2 min. (2) Photoresist stripping: photoresist stripping is performed in NMP for 10 min (simultaneous ultrasound), and then in IPA for 10 min; (3) a groove depth of a groove-type silicon wafer is 100 nm, a convex width is 70 nm, and a groove width is 100 nm.
4. A high-K dielectric HfO₂ with a thickness of 5 nm is grown on a groove-type silicon surface by an atomic layer deposition system as a gate dielectric. TEMAHf and O₃ are used as precursors during the growth, and gas-phase precursors are alternately pulsed into a reaction cavity by carrier gas (N₂) to grow at a growth temperature of 250° C.
5. ITO with a thickness of 10 nm is grown on the high-K dielectric HfO₂ by the atomic layer deposition system. An indium precursor adopted is trimethyl indium (TMIn), a tin precursor is tetrakis(dimethylamino)tin (TDMASn), an oxygen source is plasma O₂, the growth temperature is 200° C., and the ingredient proportion of indium oxide InOx to tin oxide SnOx is about 9:1. The groove depth of the groove-type ITO is 100 nm, the convex width is 100 nm, and the groove width is 70 nm.
6. The source electrode and the drain electrode are prepared by photo-leveling, exposure, development, electron beam evaporation, and stripping processes, with adopted metal being 15 nm Ni and 20 nm Au. The source electrode and the drain electrode are composed of 5-10 nm Cr and 30-50 nm Au, and the length and width of the ITO channel are 20 μm and 50 μm, respectively.
7. Process steps such as photoresist leveling, baking, exposure, development, fixing and photoresist stripping are performed to manufacture insulating layers on the source electrode and the drain electrode to isolate the source and drain electrodes from coming into contact with a test sample. (1) Spin coating SU-8 is performed with spin-coating parameters of 800 rpm/3 s, 3000 rpm/30 s, and baking is performed at 110° C. for 3 min. (2) Exposure is performed for 6 s and baking is performed at 110° C. for 2 min. (3) development with PGMEA is performed for 60 s and development with IPA is performed for 30 s. Cleaning with deionized water and blow drying with nitrogen are performed.
8. DNA probes are immobilized. (1) A device is treated with oxygen plasma to allow the surface of ITO to have hydroxyl groups, with a ratio of argon to oxygen being 4:1, the power being 15 W, and the treatment time being 5 min. (2) The device treated by oxygen plasma is immersed in an APTES solution, with the concentration of APTES being 2%, a solvent being a mixture of absolute ethanol and water, and the content of water being 5%. Reaction proceeds at room temperature for 3 hours, and after the reaction is finished, the device is cleaned with absolute ethanol and deionized water, and blow-dried with nitrogen. (3) 2 μmol/mL of DNA probe is prepared with 1×PBS buffer solution with pH=7.4, and is immobilized onto ITO by an EDC/NHS method. The concentration of EDC is 2 mmol/L, and the concentration of NHS is 10 mmol/L. Reaction proceeds in the dark at room temperature for 0.5 h. After the reaction, the device is cleaned with 1×PBS buffer solution with pH=7.4, and blow-dried with nitrogen.
9. Target DNA with different concentrations of 10 pmol/L, 100 pmol/L, and 1 nmol/L are prepared with 1×PBS buffer solution with pH=7.4. In 1×PBS buffer solution, the Debye length of an ITO interface is about 1 nm, which is much smaller than that of the DNA probe and the target DNA. First, 100 μL of 1×PBS buffer solution is added dropwise to the device and allowed to stand for 2 h. Then, 10 μL of target DNA solution is added dropwise sequentially to start the test. Test parameters are as follows: back gate voltage V_{g}=−0.1V, source-drain voltage V_{d}=50 mV, and a test channel current I_{d}-t curve. It can be seen from FIGS. 5A-5B that in the 1×PBS buffer solution with the high ionic strength, the planar ITO FET biosensor has almost no response to the target DNA, while the groove-type ITO FET biosensor can effectively overcome the influence of Debye shielding, and still has signal response to 10 pmol/L target DNA.

### Embodiment 2: Detection of COVID-19 IgG (COVID-19-IgG)

N protein (COVID-19-N) of COVID-19 is used as a probe to specifically capture COVID-19 IgG. COVID-19-N and COVID-19-IgG are purchased from Novoprotein Scientific Co., Ltd.

A groove-type field effect transistor biosensor based on an atomic layer deposited semiconductor channel comprises a substrate 1, wherein a plurality of grooves are provided at a surface of the substrate 1 in a spaced manner, a high-k dielectric layer 2 is provided on the substrate 1, an ITO channel layer 3 is provided on the high-k dielectric layer 2, source and drain electrodes 4 are provided at two ends of the ITO channel layer 3, and insulating layers 5 are provided on the source and drain electrodes 4; and the following is a preparation method of the sensor.
1. A substrate silicon is cleaned. The silicon wafer is P-type B-doped, and its resistance is less than 0.005 ohm. A standard RCA1 cleaning process is used to remove particles, organic substances, and the like on the substrate. After being cleaned, the substrate is blow-dried with high purity nitrogen for use.
2. A concave-convex silicon surface is defined by the process steps of photoresist leveling, baking, exposure, development, fixing, photoresist stripping, etc. (1) First, on the basis of step 1, spin coating with a ZEP 520A electron beam photoresist is performed with spin-coating parameters of 500 RPM/5 s and 4000 RPM/60 s, and then baking is performed at 180° C. for 3 min. (2) A groove area is defined by an electron beam exposure system. (3) Development: with a developer being xylene, development is performed for 70 s, then fixing with IPA is performed for 30 s, and blow-drying with nitrogen is performed.
3. Silicon is subjected to dry etching. (1) etching process parameters are as follows: the chuck temperature is 10° C., the pressure is 19 mtorr, the radio-frequency power is 300W, the bias voltage is 300V, the flow ratio of sulfur hexafluoride/tetracarbon octafluoride/argon gas is equal to 20/50/30 sccm, and etching is performed for 2 min. (2) Photoresist stripping: photoresist stripping is performed in NMP for 10 min (simultaneous ultrasound), and then in IPA for 10 min; (3) a groove depth of a groove-type silicon wafer is 100 nm, a convex width is 70 nm, and a groove width is 50 nm.
4. A high-K dielectric HfO₂ with a thickness of 5 nm is grown on a groove-type silicon surface by an atomic layer deposition system as a gate dielectric. TEMAHf and O₃ are used as precursors during the growth, and gas-phase precursors are alternately pulsed into a reaction cavity by carrier gas (N₂) to grow at a growth temperature of 250° C.
5. ITO with a thickness of 10 nm is grown on the high-K dielectric HfO₂ by the atomic layer deposition system. An indium precursor adopted is trimethyl indium (TMIn), a tin precursor is tetrakis(dimethylamino)tin (TDMASn), an oxygen source is plasma O₂, the growth temperature is 200° C., and the ingredient proportion of indium oxide InOx to tin oxide SnOx is about 9:1. The groove depth of the groove-type ITO is 100 nm, the convex width is 100 nm, and the groove width is 20 nm.
6. The source electrode and the drain electrode are prepared by photo-leveling, exposure, development, electron beam evaporation, and stripping processes, with adopted metal being 15 nm Ni and 20 nm Au. The source electrode and the drain electrode are composed of 5-10 am Cr and 30-50 nm Au, and the length and width of the ITO channel are 20 μm and 50 μm, respectively.
7. Process steps such as photoresist leveling, baking, exposure, development, fixing, and photoresist stripping are performed to manufacture insulating layers on the source electrode and the drain electrode to isolate the source and drain electrodes from coming into contact with a test sample. (1) Spin coating SU-8 is performed with spin-coating parameters of 800 rpm/3 s, 3000 rpm/30 s, and baking is performed at 110° C. for 3 min. (2) Exposure is performed for 6 s, and baking is performed at 110° C. for 2 min. (3) development with PGMEA is performed for 60 s, and development with IPA is performed for 30 s. Cleaning with deionized water and blow drying with nitrogen are performed.
8. COVID-19-N probes are immobilized. (1) A device is treated with oxygen plasma to allow the surface of ITO to have hydroxyl groups, with a ratio of argon to oxygen being 4:1, the power being 15 W, and the treatment time being 5 min. (2) The device treated by oxygen plasma is immersed in an APTES solution, with the concentration of APTES being 2%, a solvent being a mixture of absolute ethanol and water, and the content of water being 5%. Reaction proceeds at room temperature for 3 hours, and after the reaction is finished, the device is cleaned with absolute ethanol and deionized water, and blow-dried with nitrogen. (3) 20 μg/mL of COVID-19-N probe is prepared with the 1×PBS buffer solution with pH=7.4, and is immobilized onto ITO by the EDC/NHS method. The concentration of EDC is 2 mmol/L, and the concentration of NES is 10 mmol/L. Reaction proceeds in the dark at room temperature for 0.5 h. After the reaction, the device is cleaned with 1×PBS buffer solution with pH=7.4, and blow-dried with nitrogen. 100 μL of 2% BSA is added onto the surface of ITO dropwise, incubation is performed at room temperature for 30 min, cleaning with 1×PBS buffer solution is performed, and blow drying with nitrogen is performed for use.
9. COVID-19-IgG with different concentrations of 1 pg/mL, 10 pg/mL, and 1 ng/mL is prepared with 1×PBS buffer solution with pH=7.4. First, 100 μL of 1×PBS buffer solution is added dropwise to the device and allowed to stand for 2 h. Then, 10 μL of COVID-19-IgG solution is added dropwise sequentially to start the test. Test parameters are as follows: back gate voltage V_{g}=−0.1V, source-drain voltage V_{d}=50 mV, and a test channel current I_{d}-t curve. It can be seen from FIGS. 5A-5B that in the 1×PBS buffer solution with high ionic strength, the planar ITO FET biosensor has almost no response to the target COVID-19-IgG, which is caused by Debye shielding. As the groove-type ITO FET biosensor can effectively overcome the influence of Debye shielding, it still has signal response to 1 pg/mL COVID-19-IgG.

The applicant states that the present invention is illustrated by the above examples to show the detailed composition and method of the present invention, but the present invention is not limited to the above detailed composition and method, that is, the present invention is not meant to be necessarily dependent on the above detailed composition and method to be carried out. It should be understood by those skilled in the art that any modifications of the present invention, equivalent substitutions of the raw materials of the product of the present invention, and the addition of auxiliary components, selection of specific modes, etc., are within the scope and disclosure of the present invention.

What is clamed is:

1. A groove-type field effect transistor biosensor based on an atomic layer deposited semiconductor channel, wherein the groove-type field effect transistor biosensor comprises a substrate, a plurality of grooves are provided at a surface of the substrate in a spaced manner, a high-k dielectric layer is provided on the substrate, an indium tin oxide (ITO) channel layer is provided on the high-k dielectric layer, a source electrode and a drain electrode are provided at two ends of the ITO channel layer, and insulating layers are provided on the source electrode and the drain electrode.

2. The groove-type field effect transistor biosensor based on the atomic layer deposited semiconductor channel of claim 1, wherein the substrate is a silicon wafer, a depth of each of the plurality of grooves is 10-200 nm, a convex width is 40-200 nm, and a width of each of the plurality of grooves is 40-200 nm.

3. The groove-type field effect transistor biosensor based on the atomic layer deposited semiconductor channel of claim 1, wherein the substrate is subjected to photoresist spin coating, baking, exposure, development, fixing, dry etching, and photoresist stripping processes, or subjected to photoresist spin coating, baking, nano-imprinting, thy etching, and photoresist stripping processes, so that a flat silicon wafer is prepared into a silicon wafer substrate with the plurality of grooves provided on a surface of the silicon wafer substrate in the spaced manner.

4. The groove-type field effect transistor biosensor based on the atomic layer deposited semiconductor channel of claim 1, wherein the high-k dielectric layer is HfO₂, Al₂O₃, SiO₂, or SiNₓ, and the high-k dielectric layer is prepared by an atomic layer deposition method and has a thickness of 5-10 nm.

5. The groove-type field effect transistor biosensor based on the atomic layer deposited semiconductor channel of claim 1, wherein the ITO channel layer is prepared by an atomic layer deposition method and has a thickness of 10-20 nm, and the ITO channel layer is concave-convex and has a groove depth of 10-200 nm, a groove width of 20-300 nm, and a convex ITO width of 10-100 nm.

6. The groove-type field effect transistor biosensor based on the atomic layer deposited semiconductor channel of claim 1, wherein the source electrode and the drain electrode are one of Au, Ni/Au, and Ni/Au/Ni.

7. The groove-type field effect transistor biosensor based on the atomic layer deposited semiconductor channel of claim 1, wherein the insulating layers are SU-8, PMMA, SiO₂, or SiNₓ.

8. The groove-type field effect transistor biosensor based on the atomic layer deposited semiconductor channel of claim 1, wherein a surface of a concave-convex ITO channel layer is modified with biological probes to specifically capture target biomolecules.
